(19) United States
(12) Patent Application Publication
(10) Pub. No.: US 2020/0188652 A1
(43) Pub. Date: Jun. 18, 2020

(54) SYSTEM AND DEVICE FOR TRANSCUTANEOUS ELECTRICAL NERVE STIMULATION

(71) Applicant: Stim Spa P.C., Saratoga, CA (US)

(72) Inventors: Justin T. Wang, Saratoga, CA (US); Simone Wang, Saratoga, CA (US)

(21) Appl. No.: 16/711,117

(22) Filed: Dec. 11, 2019

Related U.S. Application Data
(60) Provisional application No. 62/779,408, filed on Dec. 13, 2018.

(51) Int. Cl. A61N 1/04; A61N 1/36

(52) U.S. Cl. CPC A61N 1/0484; A61N 1/0476; A61N 1/0452; A61N 1/0456; A61N 1/3603

(57) ABSTRACT

A system for providing electrical stimulation comprises a garment, wherein the garment includes a base material that is nonconductive and elastic. The garment may include a plurality of conductive electrodes secured to the base material, and at least one conductive path secured to the base material, wherein the at least one conductive path is configured to couple at least one conductive electrode of the plurality of conductive electrodes. The garment may include an intermediate layer disposed between at least one conductive electrode of the plurality of conductive electrodes and the base material.

### CROSS-REFERENCE TO RELATED APPLICATION

This application claims priority and benefit under 35 USC § 119(e) to U.S. Provisional Patent Application No. 62/779,408, filed on Dec. 13, 2018, which is incorporated herein by reference in its entirety.

### TECHNICAL FIELD

The present embodiments relate generally to electrical stimulation, and specifically to transcutaneous electrical nerve stimulation (TENS).

### BACKGROUND OF RELATED ART

Devices for providing electrical stimulation to the body, such as transcutaneous electrical nerve stimulation (TENS) and electrical muscle stimulation (EMS) units, are commonly used for medical and non-medical purposes. TENS uses electric current to stimulate nerves for therapeutic purposes and to reduce acute and chronic pain. In contrast, EMS uses electric current to elicit muscle contraction. EMS is commonly used for strength training, rehabilitation, testing, and post-exercise recovery. Both TENS and EMS units include a device that supplies electric current to conductive electrodes, which are strategically placed on the body.

While many TENS, EMS, and other conventional electronic stimulation devices (collectively, “conventional devices”) are available for home-market consumers, there are many disadvantages to these devices. For example, placing conductive electrodes on parts of the body that are hard to reach may be difficult. Further, many conventional devices use parts that are not standardized, and may include wires, which may limit a user's movement and agility. Conventional devices may also include detachable electrode pads and/or reservoirs of conductive material. However, these components may wear out, require replacement, and/or may be lost. Moreover, conventional devices may require additional components for conductivity, such as gel, rubber, moisture or sweat, and they may include multiple materials with different electrical resistances.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

A garment for controllably positioning electrodes for electrical stimulation is disclosed. In some aspects, the garment may include an elastic base material configured to be worn by a user. The garment may also include two or more conductive electrodes disposed on the base material and configured to contact the user's skin. The garment may also include conductive pathways printed on the base material, and configured to couple the conductive electrodes to at least two conductive buttons.

A system for providing electrical stimulation is disclosed which includes a garment, wherein the garment comprises a base material that is nonconductive and elastic; and a plurality of conductive electrodes secured to the base material, wherein each conductive electrode of the plurality of conductive electrodes comprises a plurality of conductive layers. The system may also include at least one conductive path secured to the base material, wherein the at least one conductive path is configured to couple at least two conductive electrodes of the plurality of conductive electrodes.

A system for providing electrical stimulation is disclosed. The system may include an elastic garment that may include two or more nano-silver conductive electrodes disposed onto a base material and configured to contact a user's skin and conductive pathways printed on the base material and configured to couple the conductive electrodes to at least two conductive buttons. The system may also include a control module detachably coupled to the conductive buttons and configured to provide an electrical current to the two conductive electrodes via the conductive pathways.

### BRIEF DESCRIPTION OF THE DRAWINGS

The present embodiments are illustrated by way of example and are not intended to be limited by the figures of the accompanying drawings.

FIG. 1 illustrates an example stimulation system, in accordance with some embodiments.

FIGS. 2A-2D shows block diagram depicting an example garments, in accordance with some embodiments.

FIGS. 3A-3C show an example stimulation system, in accordance with some embodiments.

FIGS. 4A-4C show another example stimulation system, in accordance with some embodiments.

FIGS. 5A-5B shows another example stimulation system, in accordance with some embodiments.

FIG. 6 shows an example garment, in accordance with some embodiments.

FIGS. 7A-7C show another example garment, in accordance with some embodiments.

FIGS. 8A-8B show another example garment, in accordance with some embodiments.

FIGS. 9A-9C show a conductive electrode integrated with base material, in accordance with some embodiments.

FIGS. 10A-10B shows an example control module, in accordance with some embodiments.

FIG. 10C shows example waveforms of a control module, in accordance with some embodiments.

FIGS. 11A-11D show example input devices, in accordance with some embodiments.

### DETAILED DESCRIPTION

As described above, conventional electrical stimulation devices have many drawbacks. Thus, there is a need for a wearable electronic stimulation device with conductive electrodes that are pre-positioned such that they may easily and repeatedly be placed on a targeted body part. There is also a need for an electronic stimulation device which consists of only a few standardized parts, and permits a user to move freely. Further, there is a need for an electronic stimulation device which does not require additional components to enhance conductivity such as gel, rubber, moisture or sweat. There is also a need for an electrical stimulation device in which the electrical resistance of the electrically conductive electrodes (also referred to as “conductive electrodes”) and electrically conductive pathways (also referred to as “conductive pathways”) is relatively constant.

Aspects of the present disclosure provide a system for providing electrical stimulation which may include a garment and a control module that is detachably coupled to the garment. The system may also include an application for an input device that communicates with the control module. The garment may include a base material and an electrically conductive component (also referred to as “conductive component”) integrated with the base material. The conductive component may include a plurality of conductive electrodes and a plurality of conductive pathways. Each conductive electrode may be configured to contact the user's skin, and each conductive pathway may be configured to couple at least one of the conductive electrodes with the control module. During operation, the control module may transmit electric current to at least one of the plurality of conductive electrodes in contact with the user's skin, thereby delivering electrical stimulation to the user's body. The system for providing electrical stimulation may be used to provide TENS, EMS, electrical massage, or any combination thereof. Furthermore, the systems described herein may be used for medical or non-medical purposes. For example, the system for electrical stimulation may be used to reduce inflammation, and/or to provide neuromodulation, pain relief, muscle and neuro stimulation, transcutaneous acupuncture, athletic rehabilitation, exercise, or athleisure. The system for electrical stimulation may also be used to stimulate synergistic acupuncture points for different conditions or indications involving sleep, headache, and/or the abdomen.

Among other advantages, the embodiments described herein provide for easy and consistent placement of the conductive electrodes against the user's body, thereby ensuring that electrical stimulation is delivered to targeted body parts. In one embodiment, an intermediate layer (e.g., a silicone or foam pad) may be disposed between the base material and the conductive electrode, thereby positioning the conductive electrode away from the garment and increasing the contact of the conductive electrode with the user's skin when the user is wearing the garment. In another embodiment, the conductive electrode may consist of multiple layers of a printed, conductive material, where the multiple layers increase the thickness of the conductive electrode. The multiple layers may position at least one surface of the conductive electrode away from the garment and increase contact pressure between the conductive electrode and the user's skin, thereby improving electrical contact when the user is wearing the garment. In yet another embodiment, the base material may include regions (e.g., strips or bands) of elastic or flexible material that help position the conductive electrodes near or on the user's skin for reliable contact. Accordingly, aspects of the present disclosure may ensure that the conductive electrodes consistently and easily contact the desired body part(s) in order to provide electrical stimulation.

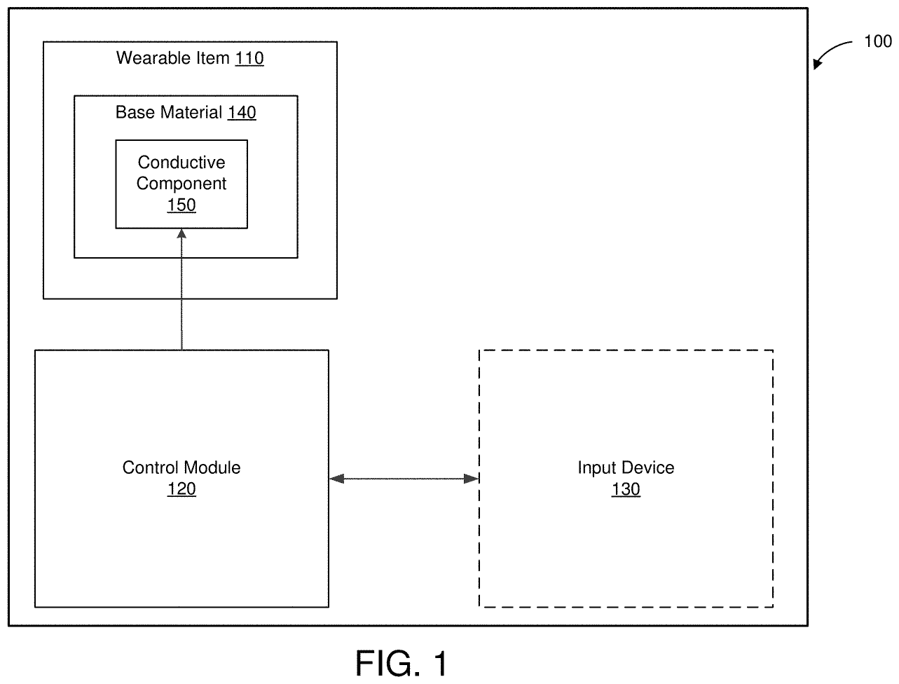

FIG. 1 illustrates an example stimulation system 100, in accordance with some embodiments. The stimulation system 100 includes a garment 110 and a control module 120. In some embodiments, the stimulation system 100 may optionally include an input device 130 (shown in dashed lines). Garment 110 may include any item designed to be worn on the body. For example, garment 110 may be any feasible clothing article such as a shirt, pants, undershirt, underpants, sock, or glove. Further, garment 110 may comprise a base material 140 and a conductive component 150. Base material 140 may be a non-conductive material that is elastic or tight-fitting, and may be designed to contact the user's skin. Base material 140 may include conductive component 150, which comprises conductive electrodes, conductive pathways, and/or conductive buttons for delivering current to specific areas of a user's body. Conductive component 150 may be controlled by control module 120. For example, control module 120 may send electric signals to conductive component 150, and may control, for example, the pulse, wavelength, and amplitude of the transmitted electric signals. The control module 120 may also communicate with input device 130. Input device 130 may be, for example, a mobile device such as a smartphone or tablet, or it may be another device such as a laptop or personal computer.

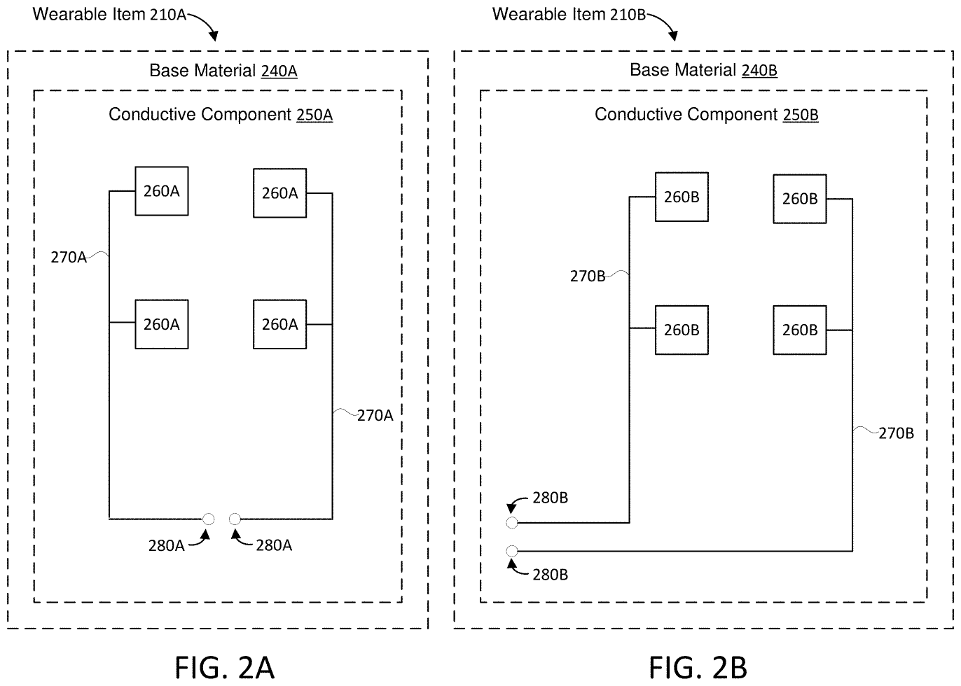

FIG. 2A shows a block diagram depicting example garment 210A, in accordance with some embodiments. The garment 210A may include base material 240A and conductive component 250A. Garment 210A may be an example embodiment of garment 110 of FIG. 1.

Base material 240A may be any item designed to be worn on the human body, or any feasible article of clothing, such as shirts with long sleeves, short sleeves or three-quarter-length sleeves, tank tops, undershirts, pants, capri pants, shorts, socks, gloves; belts, athletic wear, swimsuits, swim trunks; speedos, scarves, or undergarments. Base material 240A may be non-conductive. Further, base material 240A may be elastic, form-fitting, or tight-fitting, and may comprise similar material used for many yoga pants, leotards, leggings, bicycle shorts, and the like. As a result, when a person wears garment 210A, base material 240A lies close to the person's skin, and at least part of garment 210A may contact the person's skin

Conductive component 250A includes conductive electrodes 260A, conductive pathways 270A, and/or conductive buttons 280A. Specifically, conductive component 250A includes at least two conductive electrodes 260A. Conductive electrodes 260A may be made from any conductive material or ink (e.g., nano-silver glue), and may be configured to transmit electrical signals to the body of a person wearing garment 210A.

Relative to conductive path 270A and conductive button 280A, conductive electrode 260A may have the greatest surface area. However, this surface area may be inversely proportional to the intensity of the electric signals transmitted through conductive electrode 260A. For example, during operation, if conductive electrode 260A is relatively small, the user may feel a large amount of electrical stimulation. In contrast, if conductive electrode 260A is relatively large, the user may feel a smaller amount of electrical stimulation.

Conductive electrodes 260A may be any feasible shape (e.g., square, circular, rectangular, etc.). In one embodiment, at least one conductive electrode 260A may have a circular shape with an approximate 2-inch diameter. In another embodiment, at least one conductive electrode 260A may be square-shaped, with each side of the square measuring approximately 2 inches in length.

As discussed above, during operation, conductive electrodes 260A disposed on garment 210A may contact a person's skin. Because of this contact, there may be no need for any additional conductive medium, such as gel, rubber, sweat or moisture, disposed between the conductive electrodes 260A and the person's skin in order to enhance conductivity.

Further, in some embodiments, conductive electrodes 260A may be printed on base material 240A, on an inner surface of garment 210A. During printing, conductive electrodes 260A may be strategically placed at various positions on base material 240A in order to coincide with targeted body parts. For example, if a person desires to receive electrical stimulation to their lower back muscles, conductive electrodes 260A may be printed on base material 240A such that when a person wears garment 210A, conductive electrodes 260A contact the person's lower back muscles. As shown in FIG. 2A, conductive electrodes 260A and conductive pathways 270A may be placed in a parallel configuration. Alternatively, conductive electrodes 260A and conductive pathways 270A may be placed in a crossed configuration. If either the parallel or crossed configuration is printed on the backside of a shirt, conductive electrodes 260A may flank the lower lumbar spine and help treat lower back pain. As described further below, conductive electrodes 260A and conductive pathways 270A may be placed in any number of configurations to target different parts of the body, such as certain muscle groups or acupuncture points.

The conductive pathways 270A may be configured to transmit electric signals to conductive electrodes 260A. In some embodiments, conductive path 270A may be configured to couple at least one conductive electrode 260A with at least one conductive button 280A. As described in greater detail below, at least one conductive button 280A may be configured to connect with control module 120, which supplies the current transmitted through conductive pathways 270A to conductive electrodes 260A. Further, conductive pathways 270A may be made from the same conductive material or ink (e.g., nano-silver glue) from which conductive electrodes 260A are made. Conductive pathways 270A may be printed on base material 240A, on an inner surface or outer surface of garment 210A.

Unlike conductive electrodes 260A, conductive pathways 270A may be relatively thin, and designed to not contact the user's skin. To avoid contact, conductive pathways 270A may be disposed between at least two layers of non-conductive material, such as base material 240A. Alternatively, conductive pathways 270A may be tunneled, surrounded, or otherwise disposed between layers of non-conductive material such as base material 240A or disposed on an outer surface of the garment 210A.

As discussed above with respect to conductive electrodes 260A, the length of a conductive path 270A may affect the amount of current that passes through conductive path 270A. Accordingly, to maximize the amount of current that transmitted through the conductive electrodes 260A, the length of conductive path 270A may be minimized.

Conductive buttons 280A may be disposed at the ends of conductive pathways 270A, opposite the conductive electrodes 260A. Conductive buttons 280A may be metallic components affixed to base material 240A. For a given garment 210A, the conductive buttons 280A may be positioned close to one another, or in a central location, where conductive buttons 280A may serve as an interface between control module 120 and conductive pathways 270A and conductive electrodes 260A. Each conductive button 280A may comprise, in full or in part, a magnetic or metallic fastener. In one embodiment, a conductive button 280A may form part of a snap fastener with a disc that interlocks with a corresponding button on control module 120. This may enable control module 120 to easily and quickly be connected to or disconnected from conductive button 280A. Further, conductive buttons 280A may be affixed to any feasible location on garment 210A. In the example of FIG. 2A, the conductive buttons 280A may be positioned such that conductive buttons 280A may be centered relative to conductive electrodes 260A.

FIG. 2B shows a block diagram depicting another example garment 210B, in accordance with some embodiments. Garment 210B may include base material 240B and conductive component 250B. With reference to FIGS. 1 and 2A, garment 210B may be an embodiment of garment 110 or 210A, base material 240B may be an embodiment of base material 140 or 240A, and conductive component 250B may be an embodiment of conductive component 150 or 250A. With reference to FIG. 2A, conductive electrodes 260B may be an embodiment of conductive electrodes 260A, conductive pathways 270B may be an embodiment of conductive pathways 270A, and conductive buttons 280B may be an embodiment of conductive buttons 280A.

The garment 210B may be similar to the garment 210A of FIG. 2A. However, in the example of garment 210B, conductive buttons 280B may be positioned to one side of conductive electrodes 260B. In this configuration, if conductive electrodes 260B coincide with a user's lower back muscles, then conductive pathways 270B may extend to the opposite side of garment 210B such that the conductive buttons 280B may be positioned near the user's abdomen or waistline, within easy reach. Accordingly, when control module 120 is connected to conductive buttons 280B, a user may be able to quickly and easily connect or disconnect control module 120 from the conductive component 250B.

FIG. 2C shows a block diagram depicting another example garment 210C, in accordance with some embodiments. Garment 210C may include base material 240C and conductive component 250C. With reference to FIGS. 1 and 2A, garment 210C may be an embodiment of garment 110 or 210A, base material 240C may be an embodiment of base material 140 or 240A, and conductive component 250C may be an embodiment of conductive component 150 or 250A. With reference to FIG. 2A, conductive electrodes 260C may be an embodiment of conductive electrodes 260A, although conductive electrodes 260C may be circular, as shown in FIG. 2C. Further, conductive pathways 270C may be an embodiment of conductive pathways 270A, and conductive buttons 280C may be an embodiment of conductive buttons 280A.

FIG. 2D shows a block diagram depicting another example garment 210D, in accordance with some embodiments. Garment 210D may include base material 240D and conductive component 250D. With reference to FIGS. 1 and 2B, garment 210D may be an embodiment of garment 110 or 210B, base material 240D may be an embodiment of base material 140 or 240B, and conductive component 250D may be an embodiment of conductive component 150 or 250B. With reference to FIG. 2B, conductive electrodes 260D may be an embodiment of conductive electrodes 260B, although conductive electrodes 260D may be circular, as shown in FIG. 2D. Further, conductive pathways 270D may be an embodiment of conductive pathways 270B, and conducive buttons 280D may be an embodiment of conductive buttons 280B.

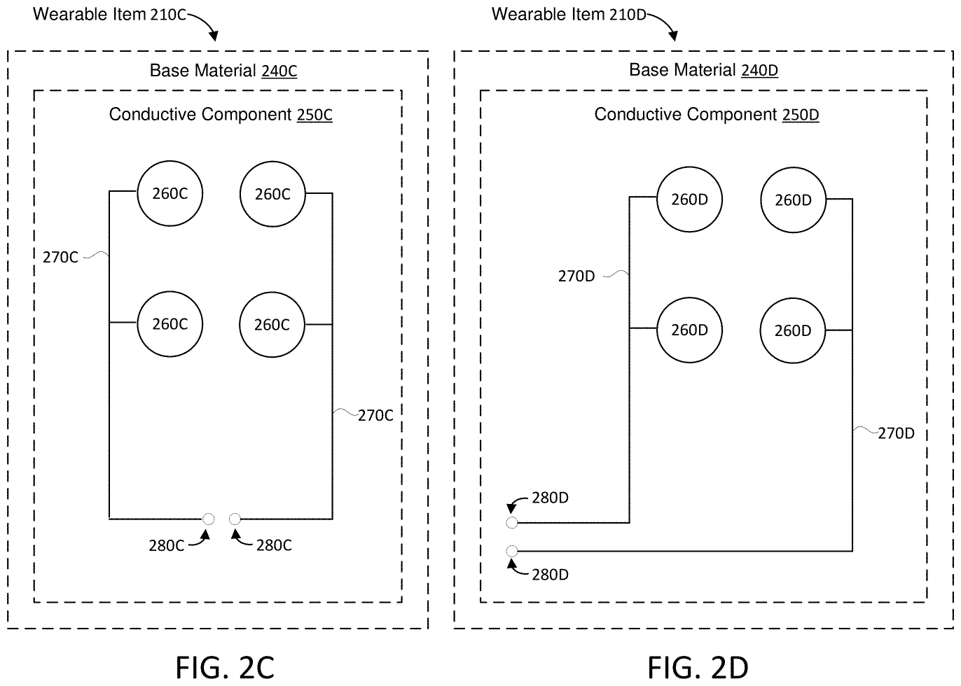

Each of FIGS. 3A-3C shows an example stimulation system 300, where garment 310 is a shirt. With reference to FIG. 1, stimulation system 300 may be an embodiment of stimulation system 100, and control module 320 may be an embodiment of control module 120. With reference to FIGS. 2A and 2B, conductive electrodes 360 may be an embodiment of conductive electrodes of 260A or 260B, and conductive pathways 370 may be an embodiment of conductive pathways 270A or 270B.

FIG. 3A shows a front view of garment 310, where control module 320 and conductive path 370 are disposed near the waistline. This configuration may permit a user wearing the garment 310 to easily and quickly access, attach, or detach control module 320 from garment 310. If control module 320 is attached to conductive path 370 via conductive buttons (not shown for simplicity), then control module 320 may be disposed on the outside or inside of garment 310. In some embodiments, control module 320 may be disposed inside of a pocket of garment 310.

FIG. 3B shows a side view of garment 310, and FIG. 3C shows a back view of garment 310. More specifically, FIG. 3C shows twelve conductive electrodes 360 coupled to conductive path 370. The twelve conductive electrodes 360 may be strategically disposed on garment 310 such that the twelve conductive electrodes 360 provide electrical stimulation to the upper, mid, and lower back regions, to muscles such as the rhomboid and trapezius, or to other muscles. While FIG. 3C depicts twelve conductive electrodes, any number of conductive electrodes may be integrated with garment 310.

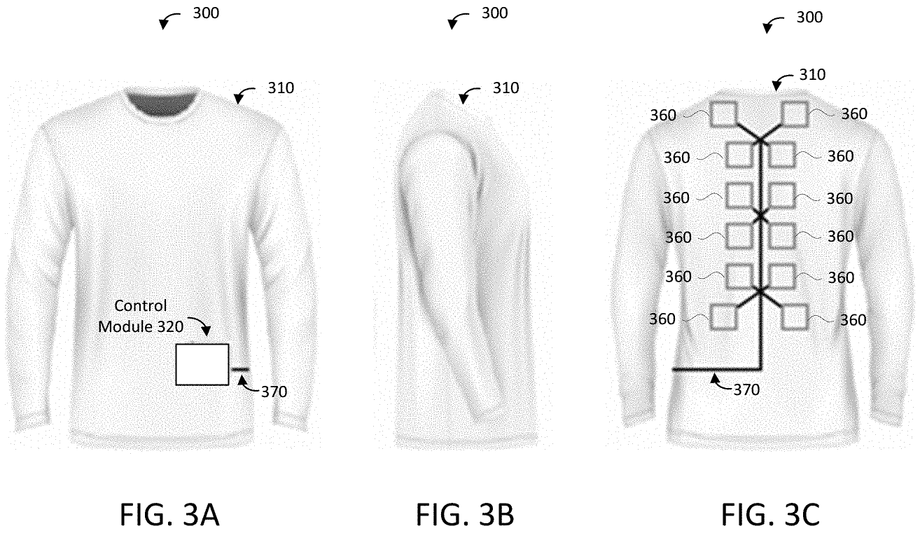

Each of FIGS. 4A-4C shows another example stimulation system 400, where garment 410 is a pair of pants. With reference to FIG. 1, stimulation system 400 may be an embodiment of stimulation system 100, and control module 420 may be an embodiment of control module 120. With reference to FIGS. 2A and 2B, conductive electrodes 460 may be an embodiment of conductive electrodes 260A or 260B, and conductive pathways 470 may be an embodiment of conductive pathways 270A or 270B.

FIG. 4A shows a front view of garment 410, where control module 420 and an end portion of conductive path 470 may be disposed near the waistline. This configuration may permit a user wearing garment 410 to easily and quickly access, attach, or detach control module 420 from garment 410. If control module 420 is attached to conductive path 470 via conductive buttons (not shown for simplicity) then control module 420 may be disposed on the outside or inside of garment 410. In some embodiments, control module 420 may be disposed inside of a pocket of garment 410. FIG. 4A also shows eight conductive electrodes 460 coupled to conductive pathways 470. The eight conductive pathways electrodes 460 may be strategically disposed on garment 410 such that the eight conductive pathways electrodes 460 may provide electrical stimulation to gluteus muscles, iliotibial (IT) bands, knee joints, the sciatic nerve, or other body parts. In the upper portion of FIG. 4A, four conductive electrodes 460 are shown along the outer thigh. These four conductive electrodes 460 may be used to treat IT band syndrome, which is common in bicyclists and runners.

FIG. 4B shows a side view of garment 410, which includes six conductive electrodes 460 and conductive path 470. FIG. 4C shows a back view of garment 410. Specifically, FIG. 4C shows twelve conductive electrodes 460 coupled to conductive path 470. The twelve conductive electrodes 460 may be strategically disposed on garment 410 such that the twelve conductive electrodes 460 provide electrical stimulation to the sacrum, sciatic nerve, gluteus muscles, IT bands, knee joints, or other body parts. While FIGS. 4A, 4B, and 4C depict example stimulation systems 400 that include eight, six, and twelve conductive electrodes 460, respectively, any number of conductive electrodes 460 may be integrated with the garment 410.

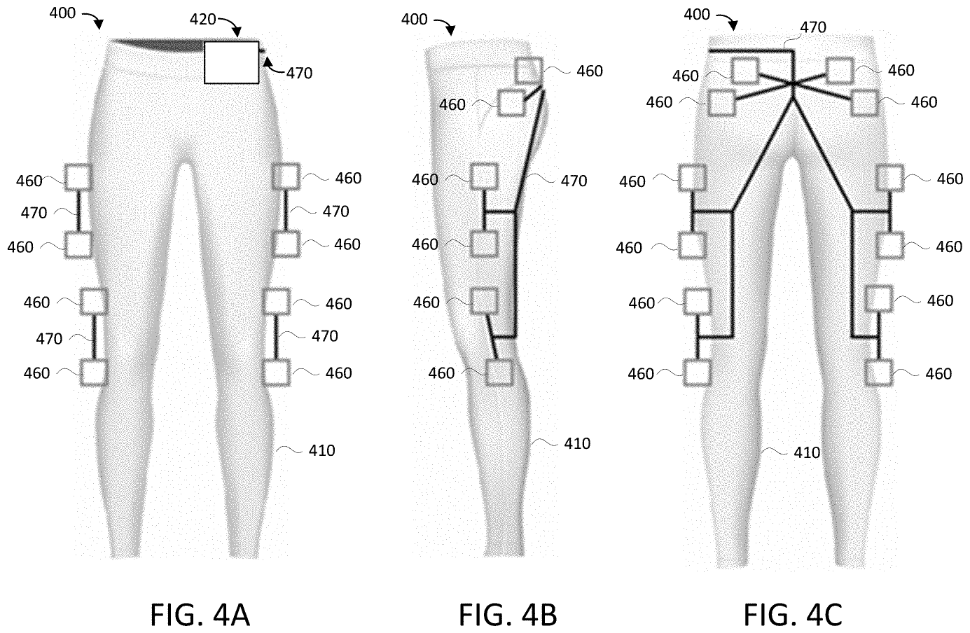

Each of FIGS. 5A and 5B shows another example stimulation system 500 and various muscles in the human body. With references to FIGS. 3A-3C and 4A-4C, stimulation system 500 may be an embodiment of stimulation system 300 or 400, control module 520 may be an embodiment of control module 320 or 420, conductive electrodes 560 may be an embodiment of conductive electrodes 360 or 460, and conductive pathways 570 may be an embodiment of conductive pathways 370 or 470. FIG. 5A shows a front view of example stimulation system 500, including eight conductive electrodes 560 coupled to conductive pathways 570 and control module 520. These eight conductive electrodes 560 may provide stimulation to the gluteus muscles, IT band, knee joints, the sciatic nerve, or other body parts. FIG. 5B shows a back view of an example stimulation system 500, including 24 conductive electrodes 560 coupled to conductive pathways 570. Conductive electrodes 560 may provide electric stimulation to the upper, mid, and lower back regions, to the rhomboid, trapezius, and gluteal muscles, and to the sacrum, sciatic nerve, IT bands, knee joints, or other body parts. While FIGS. 5A and 5B depict example stimulation systems 500 that include eight and twenty-four conductive electrodes 560, respectively, any number of conductive electrodes may be integrated in a stimulation system 500.

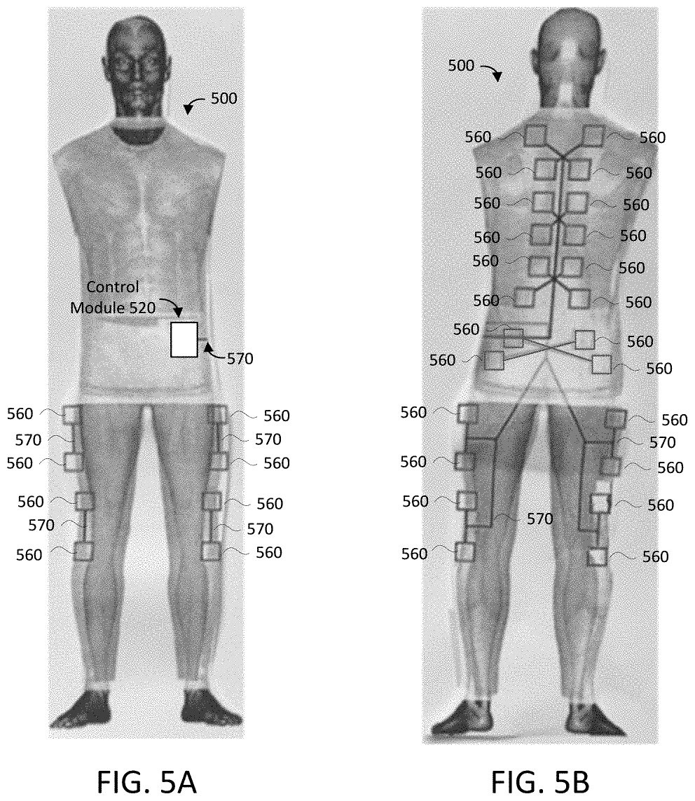

FIG. 6 shows an example garment 610, in accordance with some embodiments. With reference to FIGS. 1, 2A and 2B, garment 610 may be an embodiment of garment 110, 210A or 210B. With reference to FIGS. 2A and 2B, conductive electrodes 660 may be an embodiment of conductive electrodes 260A or 260B, and conductive path 670 may be an embodiment of conductive pathways 270A or 270B. As shown in FIG. 6, garment 610 is a shirt, and conductive electrodes 660 may be coupled to conductive path 670. Further, conductive electrodes 660 may be positioned on opposite sides of the elbow. This configuration may allow current to penetrate deep into the elbow. This configuration may also be used for other joints in the body, such as the knees or ankles.

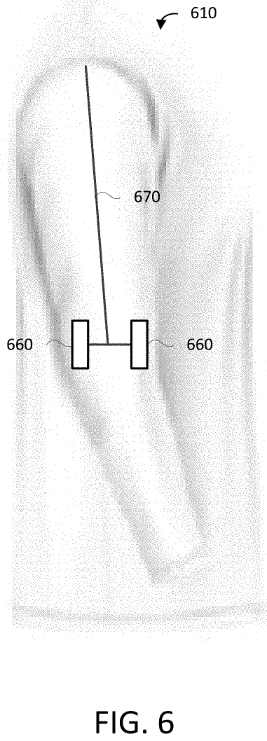

Each of FIGS. 7A-7C shows another example garment 710, in accordance with some embodiments. With reference to FIGS. 1, 2A and 2B, garment 710 may be an embodiment of garment 110, 210A or 210B, conductive electrode 760 may be an embodiment of conductive electrode 260A or 260B, and base material 740 may be an embodiment of base material 140, 240A or 240B. As described above, to maximize the electrical stimulation provided to the body, it is important that conductive electrodes contact the user's skin. In some embodiments, intermediate layer 790 may be disposed between conductive electrode 760 and base material 740, as shown in FIG. 7A. In other embodiments, intermediate layer 790 may be disposed between two layers of base material 740, as shown in FIG. 7B. Intermediate layer 790 may be any material, such as silicone or foam. The intermediate layer 790 may position or dispose the conductive electrode 760 away from the base material 740 and closer to the skin of the user. In some embodiments, if the garment 710 is not sufficiently close-fitting to the skin, then the intermediate layer 790 of FIG. 7A or multiple layers of base material 740 of FIG. 7B may help ensure contact between the conductive electrode 760 and the user.

FIG. 7C shows a view of the inside of garment 710. In some embodiments, intermediate layer 790 may be disposed between conductive electrode 760 and base material 740. Further, either a portion of or the entire perimeter of conductive electrode 760 may be attached or disposed onto base material 740. Accordingly, conductive electrode 760 and base material 740 may form a pocket surrounding intermediate layer 790. While intermediate layer 790 is shown as a circle, it may alternatively be configured as a square, rectangle, or any other feasible shape. Similarly, while conductive electrode 760 is shown as a square, it may alternatively be configured as a rectangle, circle, or any other feasible shape.

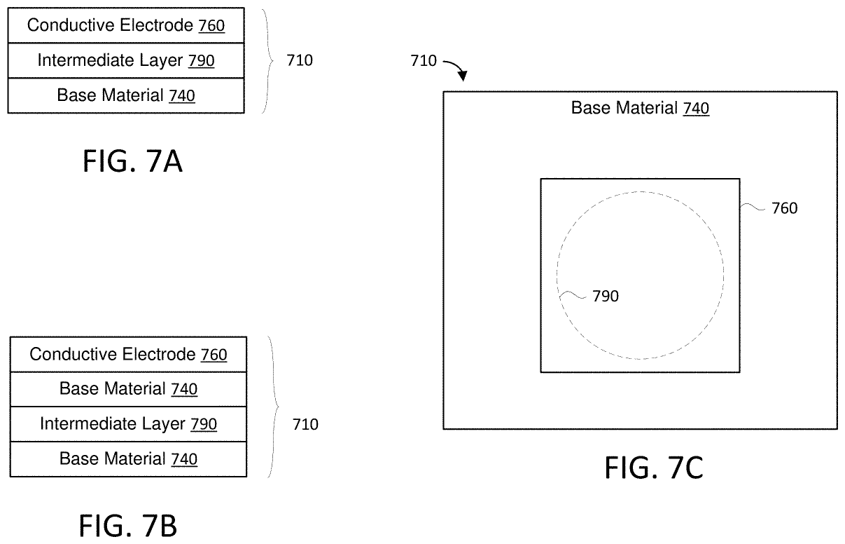

Each of FIGS. 8A and 8B shows another example garment 810, in accordance with some embodiments. With reference to FIGS. 1, 2A and 2B, garment 810 may be an embodiment of garment 110, 210A or 210B, and base material 840 may be an embodiment of base material 140, 240A or 240B. With reference to FIGS. 2A and 2B, conductive electrode 860 may be an embodiment of conductive electrode 260A or 260B. As shown in FIG. 8A, conductive electrode 860 may include multiple layers of conductive material, such as Layer 1, Layer 2, . . . Layer N. Each of Layer 1, Layer 2, . . . Layer N may comprise the same or a different conductive material. Further, the multiple layers may be printed or stacked on top of each other during manufacture. As shown in FIG. 8B, Layer 1, Layer 2, . . . Layer N may be used to increase the thickness of conductive electrode 860, thereby positioning or disposing a surface of the conductive electrode 860 away from the base material 840 and closer to the user's skin. In some embodiments, if the garment 810 is not sufficiently close-fitting to the skin, then the multiple layers of conductive material may help ensure contact between the conductive electrode 860 and the user.

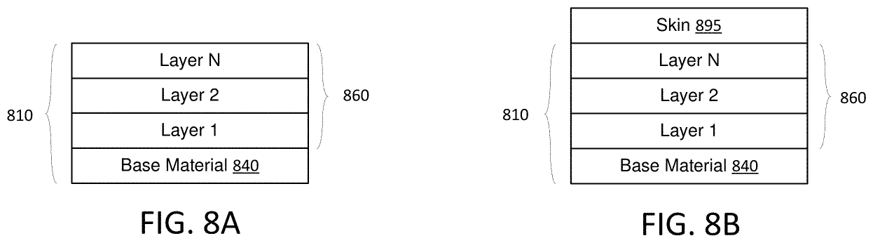

Each of FIGS. 9A-9C shows an example conductive electrode integrated with base material, in accordance with some embodiments. With reference to FIGS. 1, 2A and 2B, each garment 910A-910C may be an embodiment of garment 110, 210A, or 210B, and each base material 940A-940C may be an embodiment of base material 140, 240A or 240B. With reference to FIGS. 2A and 2B, each conductive electrode 960A-960C may be an embodiment of conductive electrode 260A or 260B.

FIGS. 9A-9C show a portion of a garment including a conductive electrode. Specifically, FIG. 9A shows a garment 910A in which an elastic strip (or band) 945A is integrated with base material 940A. Elastic band 945A may have an elasticity that is greater than that of base material 940A, and conductive electrode 960A may be attached to elastic band 945A. When garment 910A is worn by the user, elastic band 945A may have the effect of causing conductive electrode 960A to fit more closely to, or provide contact with, the user's skin. FIGS. 9B and 9C show alternative configurations with elastic bands 945B and 945C, although any number of configurations may be possible. Elastic bands 945A-945C may be rectangular, square, or any other feasible shape.

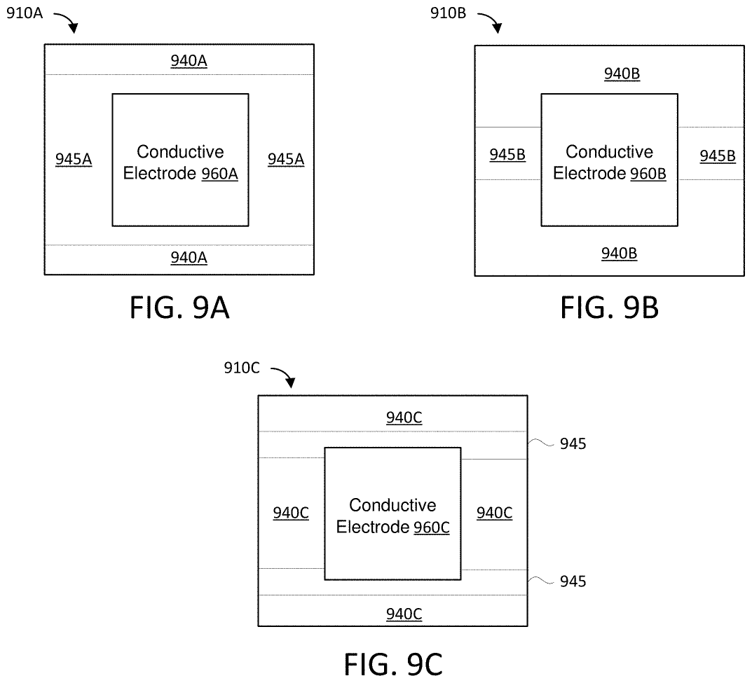

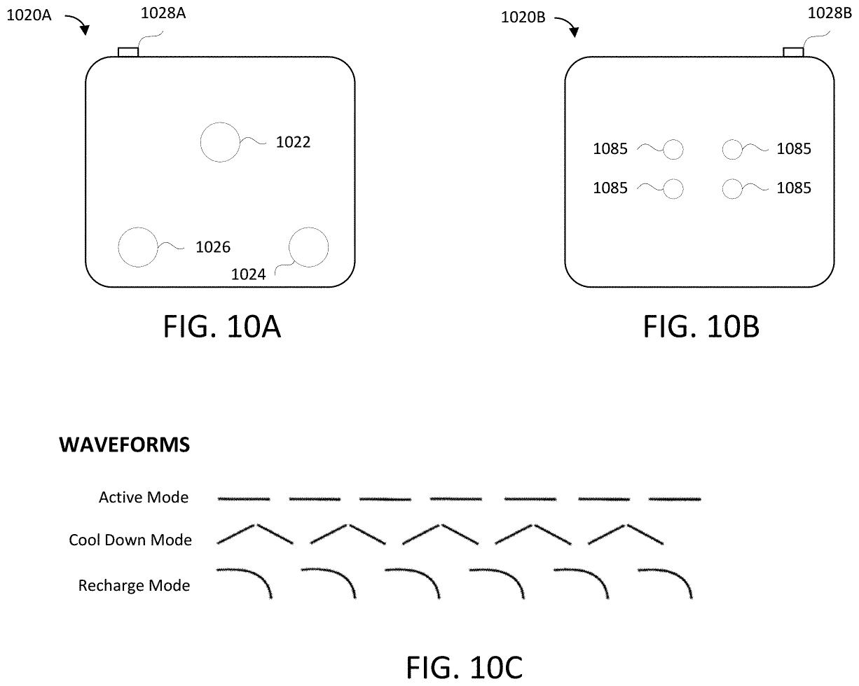

Each of FIGS. 10A and 10B shows an example control modules 1020A and 1020B, respectively, in accordance with some embodiments. With reference to FIG. 1, each of control modules 1020A and 1020B may be an embodiment of control module 120. FIG. 10A shows a front view of an example control module 1020A. Each of control modules 1020A and 1020B may supply electric current to conductive pathways and/or conductive electrodes (not shown for simplicity). Further, each of control modules 1020A and 1020B may comprise a TENs unit, EMS unit, or other control module, with preprogrammed modes. The front face of control module 1020A may include various buttons, such as buttons 1022, 1024 and 1026. Button 1022 may be used to select a particular stimulation program. Button 1024 may be used to turn on control module 1020A. Button 1026 may be used to turn off control module 1020A. Switch 1028A may be used to control the intensity of the supplied electric stimulation. While buttons 1022, 1024 and 1026 are depicted as having a circular shape, buttons 1022, 1024 and 1026 may be rectangular or any other feasible shape, and may be disposed anywhere on control module 1020A. Similarly, while switch 1028A is depicted as rectangular, switch 1028A may be circular or any other feasible shape, and may be disposed anywhere on control module 1020A.

FIG. 10B shows a back view of an example control module 1020B. With reference to FIG. 10A, control module 1020B may be an embodiment of control module 1020A, and switch 1028B may be an embodiment of switch 1028A. As shown in FIG. 10B, control module 1020B may contain one or more conductive buttons 1085, and these buttons may be disposed near each other, in a central location on the control module 1020B. Conductive buttons 1085 serve as an interface between control module 1020B and conductive electrodes disposed on a garment (not shown for simplicity). During operation, control module 1020B supplies current through conductive buttons 1085, conductive buttons, conductive pathways, and conductive electrodes of any feasible garment. In FIG. 10B, conductive buttons 1085 are shown near the center of control module 1020B; however, conductive buttons 1085 may be disposed anywhere on control module 1020B. Further, conductive buttons 1085 may comprise, in full or in part, a magnetic or metallic fastener. In one embodiment, a conductive button 1085 may form part of a snap fastener with or without a disc that interlocks with a corresponding conductive button disposed on a garment. Thus, the conductive button 1085 may enable control module 1020B to easily and quickly be connected to or disconnected from any feasible garment. A user may wish to disconnect control module 1020B from a garment to, for example, charge control module 1020B, or to wash the garment. Further, control module 1020B may be powered by a battery, such as a lithium ion battery. The battery may be recharged by a wire supplying current, or may be recharged wirelessly. While control modules 1020A and 1020B are shown as rectangular in shape in FIGS. 10A and 10B, control modules 1020A and 1020B may be square or any other feasible shape.

FIG. 10C shows example waveforms of a control module, in accordance with some embodiments. As described above, control modules 1020A and 1020B may have preprogrammed modes to provide electric stimulation. These modes may provide different forms of electric stimulation for TENS, EMS, massage, acupuncture, or other purposes. Further, each mode may correspond to electric current with different patterns, wavelengths, amplitudes, and intensities. For example, as shown in FIG. 10C, control module 1020A or 1020B may be preprogrammed with an active mode, cool down mode, or recharge mode, where each mode corresponds to electric current with a particular waveform. Control module 1020A or 1020B may also have preprogrammed modes such as burst, modulation, constant, ascending, descending, or ascending and descending. Further, control module 1020A or 1020B may have an indicator such as a light emitting diode or touch screen display. Such an indicator may use various colors or text to communicate a message or a status to the user. For example, the indicator may indicate whether control module 1020A or 1020B is on or off, the current operating mode, or the amount of charge remaining in the battery.

Control module 1020A or 1020B may supply electric current at a frequency between 1-150 Hz, and with a pulse width between 40-250 microseconds. Control module 1020A or 1020B may also supply electric current for electric stimulation that is sub-perceptive or unnoticeable to the user. Further, control module 1020A or 1020B may be configured to communicate with an input device, such as a mobile phone, via Bluetooth or another short range wireless protocol.

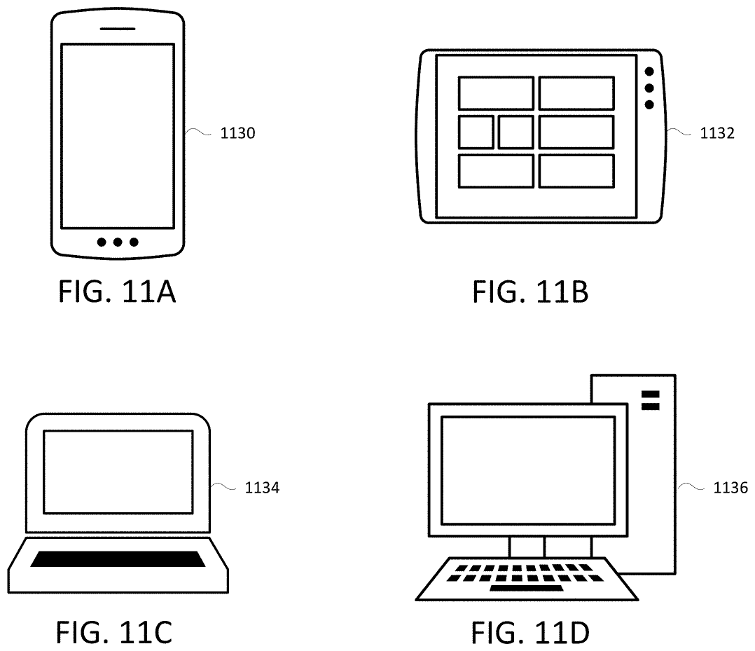

FIGS. 11A-11D show example input devices, in accordance with some embodiments. In some embodiments, the stimulation system 100 may include an application for an input device. The application may comprise software, hardware, and/or firmware. As shown in FIGS. 11A-11D, the input device may be a mobile phone or smartphone 1130 (FIG. 11A), tablet 1132 (FIG. 11B), laptop 1134 (FIG. 11C), personal computer 1136 (FIG. 11D), or the input device may be another electronic device such as a smartwatch, etc. Each of input devices 1130, 1132, 1134, and 1136 may be an embodiment of input device 130 of FIG. 1 and may use Android, iOS, or another operating system. Further, each of input devices 1130, 1132, 1134, and 1136 may communicate with control module 120 by Bluetooth or another short range communication protocol.

The application may provide an interface between any of input devices 1130, 1132, 1134, and 1136 and control module 120. Specifically, the application may allow the user to turn the control module 120 on or off, select a particular mode, select a particular region(s) of the body that is/are to receive electrical stimulation, or adjust the intensity of current supplied. Further, the application may provide an image of the body or the body parts that receive electrical stimulation. Using this image, the user may be able to select a particular body part, specify whether that body part receives stimulation, and determine what mode or intensity of stimulation may be applied to that body part. The application may also allow the user to customize certain features and set personalized treatment preferences. Additionally, the application may provide answers to frequently asked questions (FAQs), and the application may provide information relating to support help, contacting support help, and social media. The application may also enable the user to purchase items or services through an online shop.

In the foregoing specification, embodiments have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

What is claimed is:

1. A garment for controllably positioning electrodes for electrical stimulation, the garment comprising:
an elastic base material configured to be worn by a user;
two or more conductive electrodes disposed on the base material and configured to contact the user's skin; and
conductive pathways printed on the base material and configured to couple the conductive electrodes to a plurality of conductive buttons.

2. The garment of claim 1, wherein the conductive electrodes and the conductive pathways are printed onto the base material with a nano-silver glue.

3. The garment of claim 2, wherein the conductive electrodes further comprise a plurality of conductive electrodes printed upon each other and configured to dispose an outer surface of the conductive electrodes away from the base material.

4. The garment of claim 1, wherein the conductive electrodes are disposed on an intermediate layer configured to dispose the conductive electrode away from the base material.

5. The garment of claim 1, wherein the conductive electrodes are printed on an elastic band having greater elasticity than the base material.

6. The garment of claim 5, wherein the elastic band is integrated into the base material.

7. The garment of claim 1, wherein the conductive pathways are insulated from the user by the base material.

8. The garment of claim 1, wherein the conductive pathways are disposed between layers of the base material.

9. The garment of claim 1, wherein the conductive pathways are disposed on an outer surface of the base material and the conductive electrodes are disposed on an inner surface of the base material.

10. A system for providing electrical stimulation comprising:
an elastic garment comprising:
two or more conductive electrodes disposed onto a base material and configured to contact a user's skin;
conductive pathways printed on the base material and configured to couple the conductive electrodes to at least two conductive buttons; and
a control module detachably coupled to the conductive buttons and configured to provide an electrical current to the two conductive electrodes via the conductive pathways.

11. The system of claim 10, wherein the conductive electrodes are printed on the base material with a nano-silver glue.

12. The system of claim 10, wherein the conductive electrodes are disposed on an intermediate layer and configured to dispose the conductive electrodes away from the base material.

13. The system of claim 10, wherein the conductive electrodes are printed on an elastic band having greater elasticity than the base material.

14. The system of claim 13, wherein the elastic band is integrated into the base material.

15. The system of claim 10, wherein the conductive pathways are insulated from the user by the base material.

16. The system of claim 10, wherein the conductive pathways are disposed between layers of the base material.

17. The system of claim 10, wherein the conductive pathways are disposed on an outer surface of the base material and the conductive electrodes are disposed on an inner surface of the base material.
